SEQUENCE LISTING (11 sequences)

SEQ ID NO: 1 - protein, 180 aa, Artificial Sequence
  NY-ESO-1 peptide based on human sequence
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 2 - protein, 81 aa, Artificial Sequence
  XAGE-1 peptide based on human sequence
MESPKKKNQQLKVGILHLGSRQKKIRIQLRSQCATWKVICKSCISQTPGINLDLGSGVKVKIIPKEEHCKMPEAGEEQPQV

SEQ ID NO: 3 - protein, 317 aa, Artificial Sequence
  SOX2 peptide based on human sequence
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 4 - protein, 40 aa, Artificial Sequence
  NY-ESO-1 1-40, residues 1-40 of NY-ESO-1
MQAEGRGTGGSTGDADGPGGPGIPDGPGGNAGGPGEAGAT

SEQ ID NO: 5 - protein, 41 aa, Artificial Sequence
  NY-ESO-1 90-130, residues 90-130 of NY-ESO-1
FYLAMPFATPMEAELARRSLAQDAPPLPVPGVLLKEFTVSG

SEQ ID NO: 6 - protein, 41 aa, Artificial Sequence
  NY-ESO-1 120-160, residues 120-160 of NY-ESO-1
GVLLKEFTVSGNILTIRLTAADHRQLQLSISSCLQQLSLLM

SEQ ID NO: 7 - protein, 31 aa, Artificial Sequence
  NY-ESO-1 150-180, residues 150-180 of NY-ESO-1
SSCLQQLSLLMWITQCFLPVFLAQPPSGQRR

SEQ ID NO: 8 - protein, 25 aa, Artificial Sequence
  XAGE-1b 1-25, residues 1-25 of XAGE-1b
MESPKKKNQQLKVGILHLGSRQKKI

SEQ ID NO: 9 - protein, 25 aa, Artificial Sequence
  XAGE-1b 57-81, residues 57-81 of XAGE-1b
GVKVKIIPKEEHCKMPEAGEEQPQV

SEQ ID NO: 10 - protein, 31 aa, Artificial Sequence
  SOX2 52-87, residues 52-87 of SOX2
RKMAQENPKMHNSEISKRLGAEWKLLSETEK

SEQ ID NO: 11 - protein, 27 aa, Artificial Sequence
  SOX2 98-124, residues 98-124 of SOX2
RALHMKEHPDYKYRPRRKTKTLMKKDK

(19) United States
(12) Patent Application Publication
(10) Pub. No.: US 2021/0132061 A1
(43) Pub. Date: May 6, 2021

(54) Methods and Devices for Performing Auto-Antibody Assays

(71) Applicant: The Regents of the University of California, Oakland, CA (US)

(72) Inventor: Gang Zeng, Calabasas, CA (US)

(21) Appl. No.: 16/605,063

(22) PCT Filed: Apr. 13, 2018
(86) PCT No.: PCT/US2018/027534

§ 371 Date: Oct. 14, 2019

Related U.S. Application Data
(60) Provisional application No. 62/485,616, filed on Apr. 14, 2017.

(51) Int. Cl. G01N 33/564; G01N 33/543; G01N 33/68; G01N 33/574

(52) U.S. Cl. CPC G01N 33/564; G01N 33/57488; G01N 33/6854; G01N 33/543

(57) ABSTRACT

Disclosed herein are assay substrates which comprise a plurality of epitopes of one or more antigens immobilized thereon, e.g., a single microbead having more than one epitope of one or more antigens immobilized thereon, and methods of using thereof.

### ACKNOWLEDGEMENT OF GOVERNMENT SUPPORT

This invention was made with Government support under CA164388 and CA.195222, awarded by the National Institutes of Health. The Government has certain rights in the invention.

### REFERENCE TO A SEQUENCE LISTING SUBMITTED VIA EFS-WEB

The content of the ASCII text file of the sequence listing named “20180403_034044172WO1_seq_ST25” which is 8.71 kb in size was created on Apr. 3, 2018, and electronically submitted via EFS-Web herewith the application is incorporated herein by reference in its entirety.

### BACKGROUND OF THE INVENTION

### 1. Field of the Invention

The present invention generally relates to methods and devices for performing autoantibody assays.

### 2. Description of the Related Art

Recent success in immune check-point inhibitors demonstrated the power of tipping the balance of endogenously arising anti-tumor immune responses in viva. See Brahmer, et al. (2012), Garon, et al. (2015), and Topalian, et al. (2015). AutoAb response is an important arm of endogenously arising anti-tumor immune responses, and has received new attention as cancer biomarkers. See Zaenker & Ziman (2013). Due to the significant heterogeneity of tumor-associated antigens (TAA) present in cancer patients, bioniarker studies usually rely on measuring autoAb against a panel of TAA using a multiplex approach. The recent development of microbead-based multiplex assays provides a simple solution to measure autoAb responses against a panel of antigens for cancer, transplantation, infectious diseases, and others. See Xie, et al. (2011), Russo, et al. (2013), and Resse, et al. (2013). However, the requirement for purifying a large panel of recombinant proteins is difficult to achieve for most laboratories. Additionally, the loss of autoAb responses against less dominant peptide epitopes or conformational epitopes is a significant problem with the dominant epitope-based assays. See Xie, et al. (2011), Zeng, et al. (2.005), and Shih, et al. (2014).

### SUMMARY OF THE INVENTION

In some embodiments, the present invention is an assay substrate comprising a mixture of a plurality of epitopes of one or more antigens immobilized thereon. In some embodiments, the plurality of epitopes comprise at least one B-cell epitope. In some embodiments, the plurality of epitopes comprise at least one epitope of a tumor-associated antigen. In some embodiments, at least one of the one or more antigens is a tumor-associated antigen. In some embodiments, the assay substrate further includes a full-length tumor-associated antigen. In some embodiments, the full-length tumor-associated antigen is the same as the one or more antigens. In some embodiments, the full-length tumor-associated antigen is different from the one or more antigens. In some embodiments, the full-length tumor-associated antigen is XAGE-1b (SEQ ID NO: 2). In some embodiments, the full-length tumor-associated antigen is NY-ESO-1 (SEQ ID NO: 1). In some embodiments, the full-length tumor-associated antigen is SOX2 (SEQ ID NO: 3). In some embodiments, the one or more antigens include at least one of the following antigens: NY-ESO-1 (SEQ ID NO: 1), XAGE-1b (SEQ ID NO: 2), and SOX2 (SEQ ID NO: 3). In some embodiments, the one or more antigens is NY-ESO-1 (SEQ ID NO: 1). In some embodiments, the one or more antigens is XAGE-1b (SEQ ID NO: 2). In some embodiments, the one or more antigens is SOX2 (SEQ ID NO: 3). In some embodiments, at least one epitope of the plurality of epitopes is NY-ESO-1: 1-40 (SEQ ID NO: 4). In some embodiments, at least one epitope of the plurality of epitopes is XAGE-1b: 1-25 (SEQ ID NO: 8) or XAGE-1b: 57-81 (SEQ ID NO: 9). In some embodiments, at least one epitope of the plurality of epitopes is SOX2: 52-87 (SEQ ID NO: 10) or SOX2: 98-124 (SEQ ID NO: 11). In some embodiments, at least one epitope of the plurality of epitopes is SOX2: 52-87 (SEQ ID NO: 10) or SOX2: 98-124 (SEQ ID NO: 11). In some embodiments, at least one epitope of the plurality of epitopes is XAGE-1b: 1-25 (SEQ ID NO: 8) or XAGE-1b: 57-81 (SEQ ID NO: 9). In some embodiments, at least one epitope of the plurality of epitopes is NY-ESO-1: 1-40 (SEQ ID NO: 4), NY-ESO-1: 90-130 (SEQ ID NO: 5), NY-ESO-1: 120-160 (SEQ ID NO: 6), NY-ESO-1: 150-180 (SEQ ID NO: 7), XAGE-1b: 1-25 (SEQ ID NO: 8), XAGE-1b: 57-81 (SEQ ID NO: 9), SOX2: 52-87 (SEQ ID NO: 10), and/or SOX2: 98-124 (SEQ ID NO: 11). In some embodiments, the plurality of epitopes comprise, consists essentially of, or consists of at least one of the following: NY-ESO-1: 1-40 (SEQ ID NO: 4), NY-ESO-1: 90-130 (SEQ ID NO: 5), NY-ESO-1: 120-160 (SEQ ID NO: 6), NY-ESO-1: 150-180 (SEQ ID NO: 7), XAGE-1b: 1-25 (SEQ ID NO: 8), XAGE-1b: 57-81 (SEQ ID NO: 9), SOX2: 52-87 (SEQ ID NO: 10), and SOX2: 98-124 (SEQ ID NO: 11). In some embodiments, at least two epitopes of the plurality of epitopes are epitopes of the same antigen. In some embodiments, the plurality of epitopes comprise, consists essentially of, or consists of at least two of the following: NY-ESO-1: 1-40 (SEQ ID NO: 4), NY-ESO-1: 90-130 (SEQ ID NO: 5), NY-ESO-1: 120-160 (SEQ ID NO: 6), NY-ESO-1: 150-180 (SEQ ID NO: 7), XAGE-1b: 1-25 (SEQ ID NO: 8), XAGE-1b: 57-81 (SEQ ID NO: 9), SOX2: 52-87 (SEQ ID NO: 10), and SOX2: 98-124 (SEQ ID NO: 11). In some embodiments, the plurality of epitopes comprise, consists essentially of, or consists of NY-ESO-1: 1-40 (SEQ ID NO: 4) and NY-ESO-1: 90-130 (SEQ ID NO: 5). In some embodiments, the plurality of epitopes comprise, consists essentially of, or consists of NY-ESO-1: 120-160 (SEQ ID NO: 6) and NY-ESO-1: 150-180 (SEQ ID NO: 7). In some embodiments, the plurality of epitopes comprise, consists essentially of, or consists of NY-ESO-1: 1-40 (SEQ ID NO: 4), NY-ESO-1: 90-130 (SEQ ID NO: 5), and NY-ESO-1: 120-160 (SEQ ID NO: 6). In some embodiments, the plurality of epitopes comprise, consists essentially of, or consists of NY-ESO-1: 1-40 (SEQ ID NO: 4), NY-ESO-1: 90-130 (SEQ ID NO: 5), NY-ESO-1: 120-160 (SEQ ID NO: 6), and NY-ESO-1: 150-180 (SEQ ID NO: 7). In some embodiments, the plurality of epitopes comprise, consists essentially of, or consists of SOX2: 52-87 (SEQ ID NO: 10) and SOX2: 98-124 (SEQ ID NO: 11). In some embodiments, the plurality of epitopes comprise, consists essentially of, or consists of XAGE1b: 1-25 (SEQ ID NO: 8) and XAGE1b: 57-81 (SEQ ID NO: 9). In some embodiments, at least one epitope of the plurality of epitopes is NY-ESO-1: 1-40 (SEQ ID NO: 4), NY-ESO-1: 90-130 (SEQ ID NO: 5), NY-ESO-1: 120-160 (SEQ ID NO: 6), or NY-ESO-1: 150-180 (SEQ ID NO: 7). In some embodiments, the assay substrate is a microbead used in multiplex assays. In some embodiments, the assay substrate is a single spot on a substrate, e.g., microarray. In some embodiments, the assay substrate is a microwell. For example, a microwell of a microtitre plate.

In some embodiments, the present invention is an assay method for at least one antibody in a sample, which comprises contacting an assay substrate as described herein, e.g., paragraph [0011] above, with the sample, and detecting any antibodies that are specifically bound thereto. In some embodiments, the at least one antibody is an autoantibody. In some embodiments, the autoantibody specifically binds a tumor antigen. In some embodiments, the tumor antigen is expressed by prostate cancer cells. In some embodiments, the tumor antigen is expressed by lung cancer cells. In some embodiments, the tumor antigen is expressed by non-small-cell lung cancer cells.

In some embodiments, the present invention is a method for determining whether a subject has autoantibodies, which comprises contacting a sample obtained from the subject with an assay substrate as described herein, e.g., paragraph [0011] above, and detecting any antibodies that are specifically bound thereto.

In some embodiments, the present invention is a kit comprising an assay substrate as described herein, e.g., paragraph [0011] above, packaged together with one or more buffers and/or reagents for performing a detection assay with the assay substrate.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1 schematically compares a prior art multiplex microbead-based assay and a multiplex microbead-based assay according to the present invention. The prior art assay employs a plurality of different microbeads, which each microbead has only one given peptide epitope conjugated thereon, whereas the assay according to the present invention employs one microbead having a plurality of different peptides conjugated thereon. According to the inventive assay as exemplified herein, multiple confirmed peptide epitopes were conjugated to a single microbead region and diluted serum and coupled microbeads were incubated together to facilitate binding of autoAb to the peptide epitopes. A PE-labeled secondary antibody was used for the detection of a positive signal when the microbeads were analyzed individually through excitation by green and red lasers.

FIG. 2 schematically shows an embodiment where the assay substrate is a single spot, e.g., a microwell, to which antigen is immobilized thereon. The prior art employs a plurality of assay spots, which each spot has only one given peptide epitope immobilized thereon. Each assay spot of the plurality of assay spots is contacted with an aliquot of the same sample. The invention, however, provides a single assay spot that has a mixture of a plurality of different peptide epitopes conjugated thereon, which is then contacted with a test sample.

FIG. 3 to FIG. 5 graphically summarize the ELISA assays confirming the positive sera as detected by the method according to the present invention. Patient sera positive for (FIG. 3) NY-ESO-1, (FIG. 4) SOX2, and (FIG. 5) XAGE-1b were diluted 1:10, 1:20, and 1:50 and the OD at 450 nm was compared to that of control BSA for proteins NY-ESO-1 and SOX2, and control randomized peptide for XAGE-1b. Shown in the figures are all samples that have exceeded the criteria for being positive as described in the Materials and Methods herein below.

FIG. 6, Panels A and B, are Western blots confirming the positive sera as detected by the method according to the present invention. Panel A: Based on results from inventive assay exemplified herein, NY-ESO-1 sera positive patients #82, 98, and 110 were used against NY-ESO-1 transfected 293T cell lysate (L) and purified recombinant protein of NY-ESO-1 (P) on PVFD membrane. Panel B: Similarly, SOX2 sero positive patients #13 and 138 were used against SOX2 proteins (P1 and P2 from different resources) on PVDF membrane. In each panel, a positive serum was used against 293T cell lysate to show a negative reaction on far left and far right lanes labeled with N, respectively. Molecular weight for proteins were marked in kDa on the side.

### DETAILED DESCRIPTION OF THE INVENTION

In order to determine whether a full-length tumor associated antigen (TAA) may be substituted with a single dominant B-cell epitope or a plurality of B-cell epitopes in assays for autoantibodies (autoAb) against the TAA without sacrificing sensitivity and specificity, various assays employing microbeads having one or more peptides derived from cancer/testis antigens NY-ESO-1 and XAGE-1b, and transcription factor SOX2 were performed. As disclosed herein, microbead-based assays employing a plurality of B-cell epitopes immobilized on a single microbead resulted in a significant increase in sensitivity as compared to microbead-based assays employing one B-cell epitope immobilized on a given microbead.

### Mixture of Peptide Epitopes Improves Sensitivities to Detect AutoAb by Using a Single Dominant Peptide Epitope

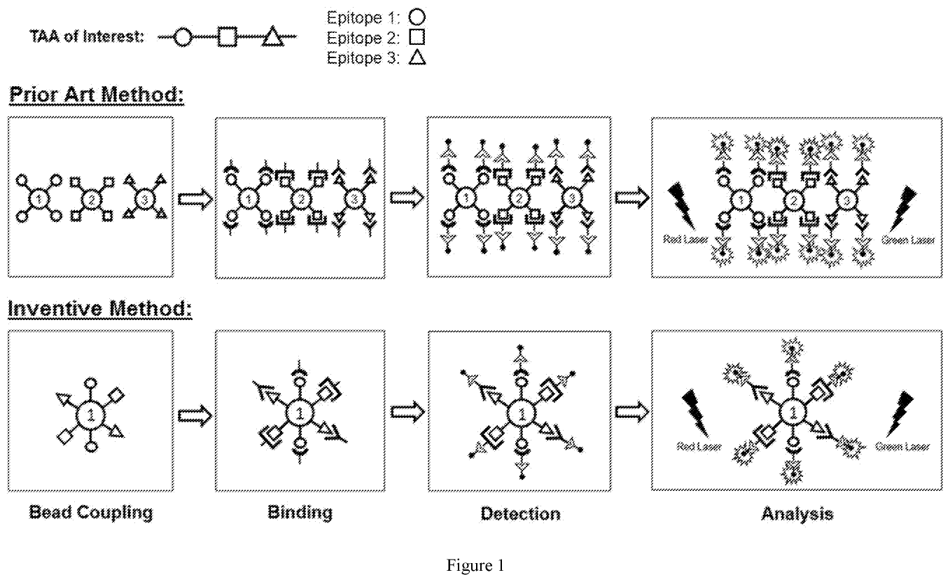

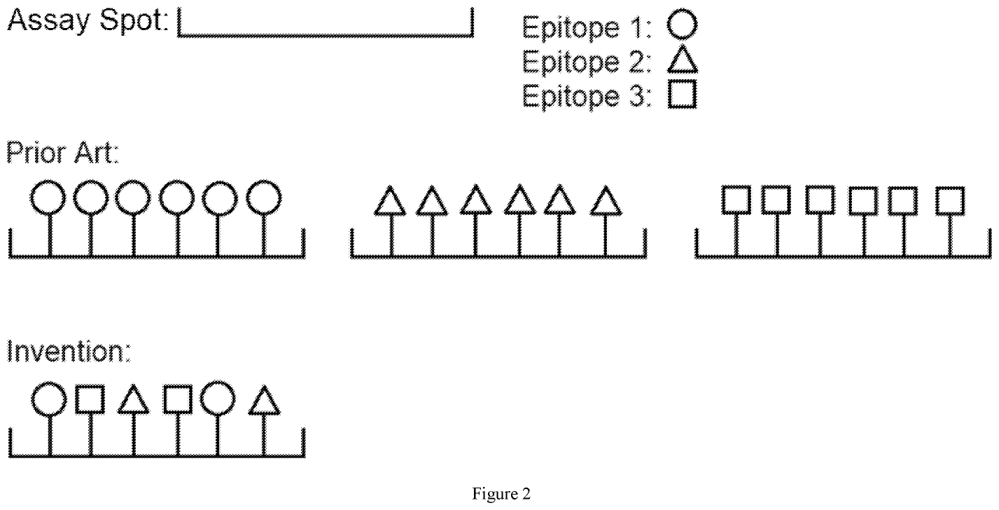

A single dominant B-cell epitope was previously used to detect autoAb response against NY-ESO-1; however, the use of a single peptide prevents the detection of autoAb against less dominant epitopes. Therefore, microbeads having a combination (e.g., mixture) of different peptide epitopes conjugated to each microbead (FIG. 1, “Inventive Method”) were compared with microbeads having only one dominant peptide epitope per microbead to determine whether microbeads having the combination of peptide epitopes is sensitive across a larger patient population. Initial experiments resulted in increased sensitivity against NY-ESO-1 and SOX2 autoAb using the mixture of peptide epitopes over single dominant peptide epitope as shown in Table 1 and Table 2.

TABLE 1
Conjugation of peptides to magnetic microbeads
and a summary of detection results

| Microbead | | | Number of |
|---|---|---|---|
| region | Analyte | Peptide epitopes (amino acids) | positive sera |
| 18 | Control | 40 mer random sequence | 0 |
| 19 | NY-ESO-1 | 1-40 (SEQ ID NO: 4) | 6 |
| 25 | NY-ESO-1 | 90-130 (SEQ ID NO: 5) | 5 |
| 26 | NY-ESO-1 | 120-160 (SEQ ID NO: 6) | 3 |
| 27 | NY-ESO-1 | 150-180 (SEQ ID NO: 7) | 4 |
| 29 | NY-ESO-1 | 1-40 (SEQ ID NO: 4); 90-130 | 14 |
| | | (SEQ ID NO: 5) | |
| 35 | NY-ESO-1 | 120-160 (SEQ ID NO: 6); | 10 |
| | | 150-180 | |
| | | (SEQ ID NO: 7) | |
| 37 | NY-ESO-1 | 1-40 (SEQ ID NO: 4); 90-130 | 7 |
| | | (SEQ ID NO: 5); 120-160 | |
| | | (SEQ ID NO: 6) | |
| 43 | NY-ESO-1 | 1-40 (SEQ ID NO: 4); 90-130 | 16 |
| | | (SEQ ID NO: 5); 120-160 | |
| | | (SEQ ID NO: 6); | |
| | | 150-180 (SEQ ID NO: 7) | |
| 45 | SOX2 | 52-87 (SEQ ID NO: 10) | 1 |
| 46 | SOX2 | 98-124 (SEQ ID NO: 11) | 2 |
| 53 | SOX2 | 52-87 (SEQ ID NO: 10); 98-124 | 4 |
| | | (SEQ ID NO: 11) | |
| 55 | XAGE-1b | 1-25 (SEQ ID NO: 8) | 4 |
| 62 | XAGE-1b | 57-81 (SEQ ID NO: 9) | 2 |
| 63 | XAGE-1b | 1-25 (SEQ ID NO: 8); 57-81 | 4 |
| | | (SEQ ID NO: 9) | |
| 65 | XAGE-1b | Full-length protein 1-81 | 11 |
| | | (SEQ ID NO: 2) | |

TABLE 2
Patients with serum positive for NY-ESO-1, SOX2,
and XAGE-1b as detected by Luminex screening

| | Analytes | | |
|---|---|---|---|
| | NY-ESO-1 | SOX2 | XAGE-1b |
| Prostate Cancer Patient | | | |
| (n = 101) | | | |
| 11 | | | + |
| 13 | + | + | + |
| 53 | + | + | |
| 62 | + | | + |
| 79 | + | + | |
| 82 | + | | |
| 98 | + | | |
| 100 | + | | |
| 107 | | | + |
| 110 | + | | + |
| 132 | + | | |
| 135 | + | | |
| 138 | | + | + |
| 139 | + | | + |
| 143 | | | + |
| 148 | + | | + |
| Total | 12 | 4 | 9 |
| Lung Cancer Patient | | | |
| (n = 32) | | | |
| 22 | | | + |
| 27 | + | | |
| 31 | + | | |
| 34 | + | | |
| 48 | + | | + |
| Total | 4 | 0 | 2 |

When only the dominant B-cell epitope, NY-ESO-1:1-40 was used, 6 patients were determined sero-positive for autoAb against NY-ESO-1. When using a mixture of peptide epitopes containing the dominant epitope, NY-ESO-1:1-40 as well as 3 other epitopes, NY-ESO-1:90-130, 120-160, and 150-180, 16 patients were determined positive for NY-ESO-1 autoAb. Similarly, increased sensitivity when using a combination of peptide epitopes was observed with SOX2. See Table 1 and Table 2.

While the incorporation of less dominant epitopes increased the sensitivity of the assay for NY-ESO-1 and SOX2, the full-length protein was the most sensitive for autoAb against for XAGE-1b. As shown in Table 1 and Table 2, the mixture of peptide epitopes detected 16 (10.6%) sera positive for NY-ESO-1 autoAb, 4 (2.7%) for SOX2, and the full-length protein detected 11 (7.3%) positives for XAGE-1b out of 151 samples screened. Due to significant aggregation, the full-length NY-ESO-1 and SOX2 proteins failed to conjugate onto the microspheres under the given conditions. Therefore, in situations where the full-length protein is unavailable or cannot be conjugated to an assay substrate, e.g., a microbead, a mixture of a plurality of fragments of the full-length protein may be conjugated to the assay substrate.

### Confirmation of Positive Sera by ELISA and Western Blot

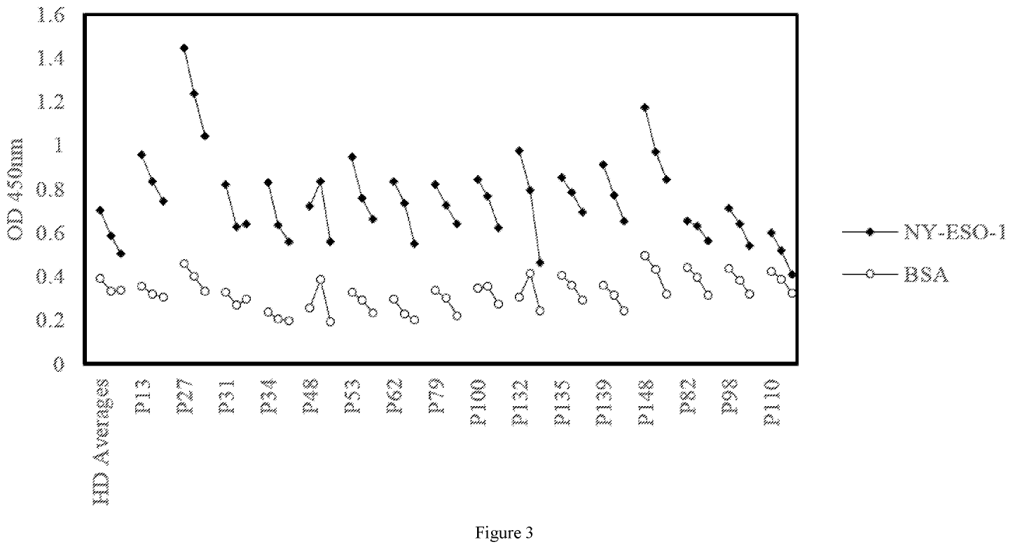

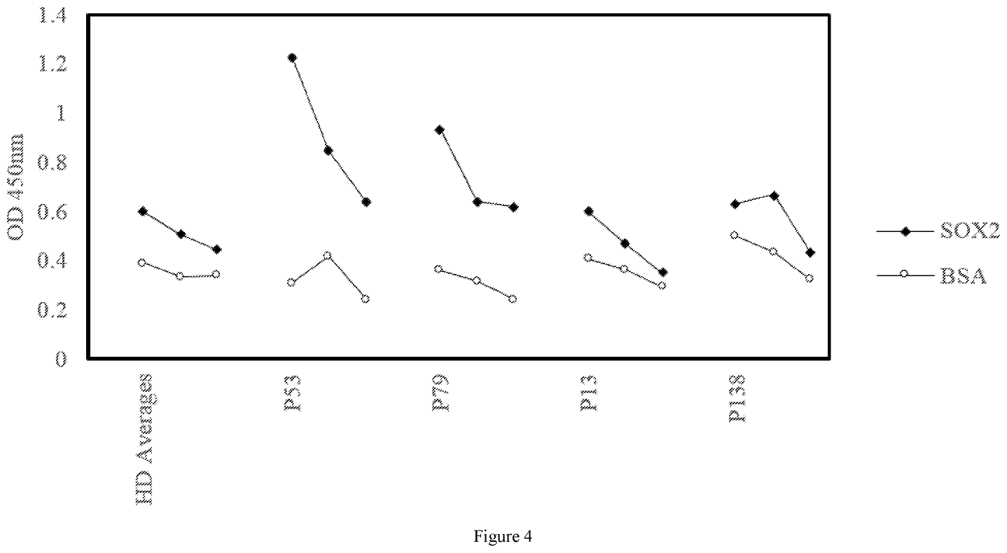

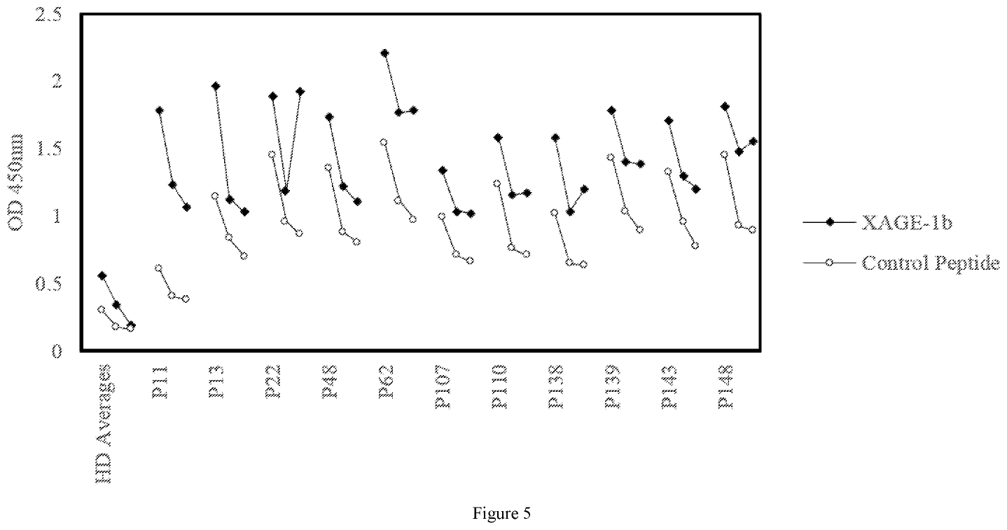

To verify the detection of autoAbs against NY-ESO-1, SOX2, and XAGE-1b as determined with microbeads having a mixture of different peptide epitopes immobilized thereon, sero-positive samples were also screened by ELISA using the full-length proteins (FIG. 3, FIG. 4, and FIG. 5). Of the 16 sera positive for autoAb against NY-ESO-1, 13 were also determined positive against the full-length NY-ESO-1 protein using ELISA (FIG. 3). About 2 out of 4 positive sera for autoAb against SOX2 were also verified against the full-length SOX2 protein by ELISA (FIG. 4), and all 11 positives for autoAb against XAGE-1b were confirmed by ELISA (FIG. 5).

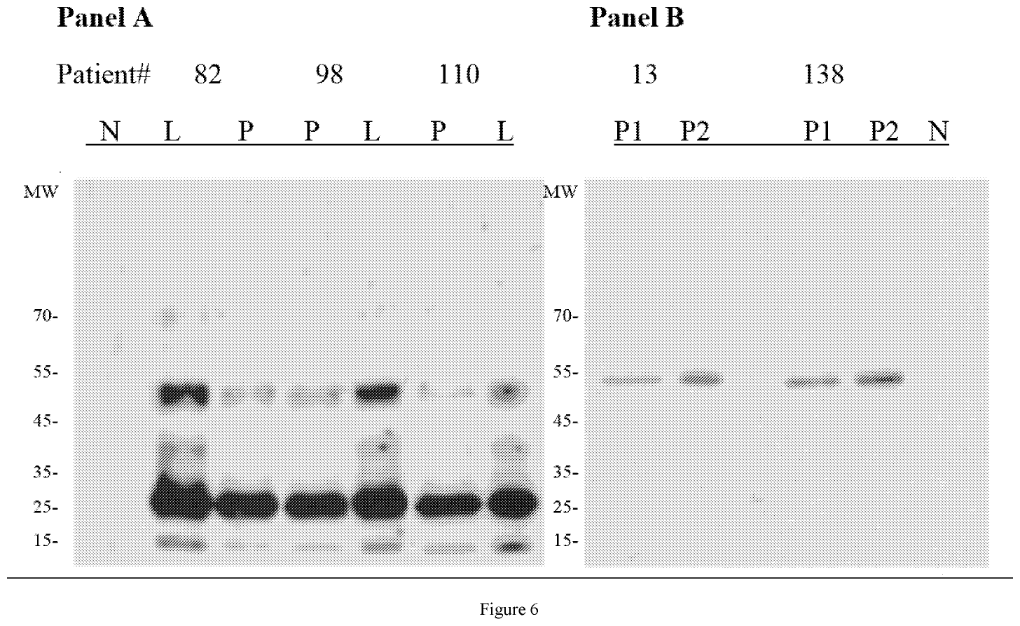

The remaining sera were then tested by Western blot. Purified protein of NY-ESO-1 and SOX2, as well as the cell lysate of transfected 293T cells for NY-ESO-1 expression and cell lysate for unaltered 293T cells, were loaded and run through SDS-PAGE. Western blots verified the presence of autoAb against NY-ESO-1 (FIG. 6, Panel A) and SOX2 (FIG. 6, Panel B) in the patient sera, and thus independently verify the presence of autoAb as detected by the assay platform—a plurality of different peptide epitopes immobilized on a single assay substrate (e.g., a single microbead)—according to the present invention.

### Discussion

The detection of autoAbs against tumor-associated antigen combined with the detection of a given tumor-specific antigen may provide improved sensitivity and specificity over assays based on detecting the tumor-specific antigen by itself. See Xie, et al. (2011), see also U.S. Pat. No. 9,354,233, which is herein incorporated by reference in its entirety. Unfortunately, prior to the present invention, assays for detecting autoAbs against TAAs have been limited. See Lagarkova, et al. (2003), Sreekumar, et al. (2004), Himoto, et al. (2005), Shi, et al. (2005), and Zhang, et al. (2001).

As disclosed herein, a mixture comprising the dominant B-cell epitope and less dominant B-cell epitopes conjugated to a single assay substrate, e.g., a single microbead, provided an unexpectedly higher level of sensitivity and specificity for autoAbs against NY-ESO-1 and SOX2 as compared to only one epitope immobilized on the single assay substrate. These results are surprising because many TAAs have more than one epitope that are recognized by the given autoAb such that one would expect substantially similar assay results, or, alternatively, a decrease in sensitivity and/or specificity as a result of interference between different epitopes being in close proximity by being immobilized together on the same assay substrate.

Although the mixture of peptide epitopes does not seem to be as effective when compared to the full-length protein, as in the case of XAGE-1b, when the full-length protein is not available or cannot be conjugated to an assay substrate, as in the case of NY-ESO-1 and SOX2, a mixture comprising a plurality of epitopes from the full-length protein may be used as a substitute for the full-length protein itself.

Because a plurality of different epitopes may be conjugated to a single assay substrate, such as a microbead, without a negative impact on assay sensitivity or specificity, in some embodiments, epitopes of two or more different TAAs may be likewise conjugated on a single assay substrate to provide one diagnostic device that can be used to detect one or more autoAb. For example, at least one epitope of a first protein and at least one epitope from a second protein may be conjugated on the same assay substrate, which can then be used in detection assays for antibodies against one or both proteins in a given sample. As another example, at least one epitope of a first TAA, and at least one epitope of a second TAA, wherein the first TAA and the second TAA are different, may be immobilized on a single assay substrate, such as a microbead, and used to detect autoAbs against one or both TAAs in a sample from a subject. The presence or absence of one or both TAAs may then be used as diagnosing a subject as having a given cancer. In some embodiments, the first TAA, the second TAA, or both include one or more epitopes of NY-ESO-1, SOX2, and/or XAGE-1b.

The following examples are intended to illustrate but not to limit the invention.

### Materials and Methods

Protein Sequences
NY-ESO-1:
SEQ ID NO: 1:
MQAEGRGTGGSTGDADGPGGPGIPDGPGGNAGGPGEAGATGGRGPRGAGA
ARASGPGGGAPRGPHGGAASGLNGCCRCGARGPESRLLEFYLAMPFATPM
EAELARRSLAQDAPPLPVPGVLLKEFTVSGNILTIRLTAADHRQLQLSIS
SCLQQLSLLMWITQCFLPVFLAQPPSGQRR
XAGE-1:
SEQ ID NO: 2:
MESPKKKNQQLKVGILHLGSRQKKIRIQLRSQCATWKVICKSCISQTPGI
NLDLGSGVKVKIIPKEEHCKMPEAGEEQPQV
SOX2:
SEQ ID NO: 3:
MYNMMETELKPPGPQQTSGGGGGNSTAAAAGGNQKNSPDRVKRPMNAFMV
WSRGQRRKMAQENPKMHNSEISKRLGAEWKLLSETEKRPFIDEAKRLRAL
HMKEHPDYKYRPRRKTKTLMKKDKYTLPGGLLAPGGNSMASGVGVGAGLG
AGVNQRMDSYAHMNGWSNGSYSMMQDQLGYPQHPGLNAHGAAQMQPMHRY
DVSALQYNSMTSSQTYMNGSPTYSMSYSQQGTPGMALGSMGSVVKSEASS
SPPVVTSSSHSRAPCQAGDLRDMISMYLPGAEVPEPAAPSRLHMSQHYQS
GPVPGTAINGTLPLSHM

As provided herein, epitopes of the above-referenced proteins are indicated by the particular span of amino acid residues. For example, NY-ESO-1: 1-40 is the amino acid sequence from the 1st amino acid residue to the 40th amino acid residue of NY-ESO-1. Therefore, the following peptide epitopes are exemplified herein:

NY-ESO-1: 1-40
SEQ ID NO: 4: MQAEGRGTGGSTGDADGPGGPGIPDGPGGNAGGPGE
AGAT
NY-ESO-1: 90-130
SEQ ID NO: 5: FYLAMPFATPMEAELARRSLAQDAPPLPVPGVLLKE
FTVSG
NY-ESO-1: 120-160
SEQ ID NO: 6: GVLLKEFTVSGNILTIRLTAADHRQLQLSISSCLQQ
LSLLM
NY-ESO-1: 150-180
SEQ ID NO: 7: SSCLQQLSLLMWITQCFLPVFLAQPPSGQRR
XAGE 1b: 1-25
SEQ ID NO: 8: MESPKKKNQQLKVGILHLGSRQKKI
XAGE 1b: 57-81
SEQ ID NO: 9: GVKVKIIPKEEHCKMPEAGEEQPQV
SOX2: 52-87
SEQ ID NO: 10: RKMAQENPKMHNSEISKRLGAEWKLLSETEK
SOX2: 98-124
SEQ ID NO: 11: RALHMKEHPDYKYRPRRKTKTLMKKDK

### Serum Samples

Serum samples were collected under institutional review board-approved protocols from UCLA and collaborating hospitals, and stored at −80° C. until use. Serum samples from healthy donors (HD) were obtained from subjects routinely screened to exclude the presence of concomitant disease and cancer patient serum samples were collected at time of biopsy and prior to surgery (Table 3). Positive controls were based on previous screening results. See Xie, et al. (2011).

TABLE 3
Serum samples used in autoAb studies.

| Sample Type | Total number of sera |
|---|---|
| Prostate Cancer | 101 |
| Lung Cancer | 32 |
| Healthy Donor | 8 |
| Positive Control to | 10 |
| NY-ESO-1 | 4 |
| SOX2 | 2 |
| XAGE-1b | 4 |
| Total | 151 |

### Microbead-Based Assays

Serum samples were screened using Luminex® microsphere-based assays (Austin, Tex.). Peptide epitopes from prototype antigens NY-ESO-1, SOX2, XAGE-1b (Genemed Synthesis, San Antonio, Tex.), and a control random peptide sequence (Genscript, Piscataway, N.J.) were conjugated onto microbeads (Bio-Rad, Hercules, Calif.) by using sulfo-NHS (Thermo Fisher, Waltham, Mass.) to convert carboxyl groups on the microbeads to amine-reactive esters, and EDC (Thermo Fisher) to couple the ester to primary amine groups on the peptides (FIG. 1). Peptides were conjugated onto the microbeads at 20 μg per 1.0×10⁶ microspheres (Table 1).

Serum samples were diluted at 1:10, 1:50, and 1:200 in assay buffer (PBS, 1% BSA) and incubated with 2500 beads per region of conjugated microspheres in 96-well plates (Bio-Rad) for 1 hour at room temperature with gentle agitation. The plates were then washed 3 times with wash buffer (PBS, 1.0% Bovine Serum Albumin, 0.1% Sodium azide, 0.05% Tween-20) using a magnetic plate washer (Bio-Rad). Secondary antibody, PE conjugated goat anti-human IgG (Jackson ImmunoResearch, West Grove, Pa.), was added to the wells and incubated for 1 hour at room temperature with gentle agitation. The plate was once again washed 3 times with wash buffer and fluorescence intensity (FI) was recorded using the Bio-Plex 200 system (Bio-Rad). FI of auto-antigen epitope-conjugated microspheres were compared to those of a baseline random peptide control by taking the ratio of fluorescence intensity (RFI). The RFI of patient serums were compared to those of healthy donor serum, and a positive reaction was defined as RFIs that were 3 standard deviations above the mean.

### ELISA

Positive serum samples detected by Luminex screening were confirmed using ELISA. Antigen-coated Nunc ELISA plates (eBioscience, San Diego, Calif.) were prepared using 50 ng/well of purified NY-ESO-1, 250 ng/well SOX2 protein, and 100 ng/well control BSA protein in 100 μL of carbonate bicarbonate buffer. ELISA plates were also coated with 60 ng/well of full-length XAGE-1b peptide and 60 ng/well control randomized synthetic peptide in 100 μL of carbonate bicarbonate buffer. Coated plates were then left to incubate overnight at 4° C. to allow the absorbance of the antigens into the plates. Plates were blocked with 5% fetal bovine serum (FBS) in PBS+0.05% Tween 20 (PBST) for 2 hours and then washed with PB ST, and loaded with 100 μL of diluted serum samples. Serum samples were pre-diluted at 1:10, 1:20, and 1:50 with 5% FBS in PBST. The serum samples, once loaded onto the pre-coated ELISA plates, were left to incubate for 2 hours at room temperature, after which the plates were washed again with PBST, and then loaded with secondary antibody of goat anti-human immunoglobulin conjugated with horseradish peroxidase (Sigma Aldrich, St. Louis, Mo.) diluted with 5% FBS in PB ST. Plates were developed after 1 hour of incubation, after which absorbance (OD) at 450 nm was recorded. The difference in OD was calculated by subtracting the BSA protein or control peptide from the OD of the proteins or peptide of interest: NY-ESO-1 protein, SOX2 protein, and XAGE-1b full-length protein. Positive reactions were defined as ΔOD that were 3 standard deviations above the mean.

### Western Blot

Serum samples that were positive for autoantibody response as determined by Luminex, but not confirmed by ELISA, were tested by Western blotting. Purified NY-ESO-1 protein and NY-ESO-1 expressing cell lysates from transfected 293T cells as well as purified proteins of SOX2 from 2 sources (Genscript and Genemed Synthesis), were run on 4-12% Bis-Tris SDS gels (Thermo Fisher) alongside negative control 293T cell lysate. Following separation, the proteins were transferred to PVDF membrane (Thermo Fisher). The membrane was blocked using 5% milk in PB ST overnight at 4° C. Membrane was washed with PBST and then incubated for 1 hour at room temperature with serum samples diluted 1:1000 in blocking buffer (5% milk in PBST, 0.1% SDS was added to reduce reaction background). Secondary antibody, HRP conjugated goat anti-human IgG (Jackson ImmunoResearch), was diluted in 5% milk in PBST and applied to the membrane for 1 hour at room temperature. The membrane was developed using ECL Western Development Kit (Thermo Fisher).

### REFERENCES

1. Brahmer, et al. (2012)
   Safety and activity of anti-PD-L1 antibody in patients with advanced cancer. N Engl J Med 366: 2455-2465.
2. Garon, et al. (2015)
   Pembrolizumab for the treatment of non-small-cell lung cancer. N Engl J Med 372: 2018-2028.
3. Topalian, et al. (2015)
   Immune checkpoint blockade: a common denominator approach to cancer therapy. Cancer Cell 27: 450-461.
4. Zaenker & Ziman (2013)
   Serologic autoantibodies as diagnostic cancer biomarkers-a review. Cancer Epidemiol Biomarkers Prey 22: 2161-2181.
5. Xie, et al. (2011)
   A Novel Multiplex Assay Combining Autoantibodies Plus PSA Has Potential Implications for Classification of Prostate Cancer from Non-malignant Cases. J Transl Med 9: 43.
6. Russo, et al. (2013)
   A novel approach to biomarker discovery in head and neck cancer using an autoantibody signature. Oncogene 32: 5026-5037.
7. Resse, et al. (2013)
   Anti-HLA-A, -B, -DR, -DQB1 and -DQA1 antibodies reactive epitope determination with HLAMatchmaker in multipare awaiting list for heart transplant. Hum Immunol 74: 937-941.
8. Zeng, et al. (2005)
   Dominant B cell epitope from NY-ESO-1 recognized by sera from a wide spectrum of cancer patients: implications as a potential biomarker. Int J Cancer 114: 268-273.
9. Shih, et al. (2014)
   Dominant B-cell epitopes from cancer/stem cell antigen SOX2 recognized by serum samples from cancer patients. American Journal of Clinical and Experimental Immunology 3: 84-90.
10. Simpson, et al. (2005)
   Cancer/testis antigens, gametogenesis and cancer. Nat Rev Cancer 5: 615-625.
11. Lagarkova, et al. (2003)
   Evaluation of humoral response to tumor antigens using recombinant expression-based serological mini-arrays (SMARTA). Immunol Lett 85: 71-74.
12. Sreekumar, et al. (2004)
   Humoral immune response to alpha-methylacyl-CoA racemase and prostate cancer. J Natl Cancer Inst 96: 834-843.
13. Himoto, et al. (2005)
   Analyses of autoantibodies against tumor-associated antigens in patients with hepatocellular carcinoma. Int J Oncol 27: 1079-1085.
14. Shi, et al. (2005)
   Preferential humoral immune response in prostate cancer to cellular proteins p90 and p62 in a panel of tumor-associated antigens. Prostate 63: 252-258.
15. Zhang, et al. (2001)
   De-novo humoral immune responses to cancer-associated autoantigens during transition from chronic liver disease to hepatocellular carcinoma. Clin Exp Immunol 125: 3-9.

All scientific and technical terms used in this application have meanings commonly used in the art unless otherwise specified.

The use of the singular can include the plural unless specifically stated otherwise. As used in the specification and the appended claims, the singular forms “a”, “an”, and “the” can include plural referents unless the context clearly dictates otherwise. The use of “or” can mean “and/or” unless stated otherwise. As used herein, “and/or” means “and” or “or”. For example, “A and/or B” means “A, B, or both A and B” and “A, B, C, and/or D” means “A, B, C, D, or a combination thereof” and said “combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, an “epitope” is the part of a molecule that is recognized by a given antibody.

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus.

As used herein, “autoantibody” refers to an antibody produced by a subject that is directed against one or more of the subject's own antigens (e.g., a tumor-associated antigen). As used herein, “antibody” refers to naturally occurring and synthetic immunoglobulin molecules and immunologically active portions thereof (i.e., molecules that contain an antigen binding site that specifically bind the molecule to which antibody is directed against). As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include, but are not limited to: single chain Fvs (scFvs), Fab fragments, Fab' fragments, F(ab')2, disulfide linked Fvs (sdFvs), Fvs, and fragments comprising or alternatively consisting of, either a VL or a VH domain.

As used herein, a molecule, e.g., an antibody, that “specifically binds” another molecule, means that the interaction is dependent upon the presence of a specific structure, e.g., an epitope, on the molecule being bound. For example, an antibody which specifically binds a protein is recognizing and binding a specific structure on the protein rather than indiscriminate binding that gives rise to non-specific binding and/or background binding. As used herein, “non-specific binding” and “background binding” refer to an interaction that is not dependent on the presence of a specific structure (e.g., a particular epitope).

As used herein, “tumor antigens” refer to tumor-specific antigens (TSAs), which generally classified as antigens present only on tumor cells and tumor-associated antigens (TAAs), which are generally classified as antigens present on some tumor cells and also some normal cells.

As used herein, the term “subject” includes humans and non-human animals. The term “non-human animal” includes all vertebrates, e.g., mammals and non-mammals, such as non-human primates, horses, sheep, dogs, cows, pigs, chickens, and other veterinary subjects and test animals. As used herein, the term “subject” may be used interchangeably with “patient”.

As used herein, the term “sample” is used in its broadest sense and includes specimens and cultures obtained from any source, as well as biological and environmental samples. Biological samples may be obtained from animals (including humans) and encompass fluids, solids, tissues, and gases. Biological samples include blood products, such as plasma, serum, and the like.

As used herein, a “capture reagent” refers to a molecule which specifically binds with an analyte of interest. The capture reagent may be immobilized on a substrate. For example, if the analyte of interest is an antibody, the capture reagent may be an antigen or an epitope thereof to which the antibody specifically binds.

As used herein, an “assay substrate” refers to any substrate that may be used to immobilize a capture reagent thereon and then detect an analyte when bound thereto.

As used herein, a “detectable label” is a compound or composition that produces or can be induced to produce a signal that is detectable by, e.g., visual, spectroscopic, photochemical, biochemical, immunochemical, or chemical means. The use of the term “labeled” as a modifier of a given substance, e.g., a labeled antibody, means that the substance has a detectable label added thereto. A detectable label can be attached directly or indirectly by way of a linker (e.g., an amino acid linker or a chemical moiety). Examples of detectable labels include radioactive and non-radioactive isotopes (e.g., ¹²⁵I, ¹⁸F, ^{C}, etc.), enzymes (e.g., β-galactosidase, peroxidase, etc.) and fragments thereof, enzyme substrates, enzyme inhibitors, coenzymes, catalysts, fluorophores (e.g., rhodamine, fluorescein isothiocyanate, etc.), dyes, chemiluminescers and luminescers (e.g., dioxetanes, luciferin, etc.), and sensitizers.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. An assay substrate comprising a mixture of a plurality of epitopes of one or more antigens immobilized thereon.

2. The assay substrate according to claim 1, wherein the plurality of epitopes comprise at least one B-cell epitope.

3. The assay substrate according to claim 1, wherein the plurality of epitopes comprise at least one epitope of a tumor-associated antigen.

4. The assay substrate according to claim 1, wherein at least one of the one or more antigens is a tumor-associated antigen.

5. The assay substrate according to claim 1, and further including a full-length tumor-associated antigen which may be the same as or different from the one or more antigens.

6. The assay substrate according to claim 1, wherein the one or more antigens include at least one of the following antigens: NY-ESO-1 (SEQ ID NO: 1), XAGE-1b (SEQ ID NO: 2), and SOX2 (SEQ ID NO: 3).

7. The assay substrate according to claim 5, wherein the full-length tumor-associated antigen is XAGE-1b (SEQ ID NO: 2).

8. The assay substrate according to claim 1, wherein at least one epitope of the plurality of epitopes is NY-ESO-1: 1-40 (SEQ ID NO: 4).

9. The assay substrate according to claim 1, wherein at least one epitope of the plurality of epitopes is NY-ESO-1: 1-40 (SEQ ID NO: 4), NY-ESO-1: 90-130 (SEQ ID NO: 5), NY-ESO-1: 120-160 (SEQ ID NO: 6), or NY-ESO-1: 150-180 (SEQ ID NO: 7).

10. The assay substrate according to claim 1, wherein at least one epitope of the plurality of epitopes is SOX2: 52-87 (SEQ ID NO: 10) or SOX2: 98-124 (SEQ ID NO: 11).

11. The assay substrate according to claim 1, wherein at least one epitope of the plurality of epitopes is XAGE-1b: 1-25 (SEQ ID NO: 7) or XAGE-1b: 57-81 (SEQ ID NO: 8).

12. The assay substrate according to claim 1, wherein at least two epitopes of the plurality of epitopes are epitopes of the same antigen.

13. The assay substrate according to claim 1, wherein the assay substrate is a microwell or a microbead.

14. The assay substrate according to claim 1, wherein the assay substrate is a microbead used in multiplex assays.

15. An assay method for at least one antibody in a sample, which comprises contacting the assay substrate according to claim 1 with the sample, and detecting any antibodies that are specifically bound thereto.

16. The assay method according to claim 15, wherein the at least one antibody is an autoantibody.

17. A method for determining whether a subject has autoantibodies, which comprises contacting a sample obtained from the subject with the assay substrate according to claim 1, and detecting any antibodies that are specifically bound thereto.

18. A kit comprising an assay substrate according to claim 1 packaged together with one or more buffers and/or reagents for performing a detection assay with the assay substrate.
